(19) United States
(12) Patent Application Publication
(10) Pub. No.: US 2015/0007812 A1
(43) Pub. Date: Jan. 8, 2015

(54) Delivery of Nebulized Medicines

(71) Applicant: Mathew S. Smith, Bend, OR (US)

(72) Inventor: Mathew S. Smith, Bend, OR (US)

(21) Appl. No.: 13/934,565

(22) Filed: Jul. 3, 2013

(51) Int. Cl. A61M 11/02

(52) U.S. Cl. CPC A61M 11/02
USPC 128/200.21

(57) ABSTRACT

Devices are disclosed that include an elongated member configured to support a delivery tube and maintain a distal end of the tube in a desired position, and to allow the position of the distal end of the tube to be adjusted by a user, and a base configured to support a proximal end of the elongated member. The base includes a chamber configured to receive a medicine cup containing a supply of medicine and position a first end of the medicine cup in fluid communication with a source of compressed gas and a second end of the medicine cup in fluid communication with the delivery tube. These devices may be used, for example, in the delivery of nebulized medicine to a patient, e.g., a child or infant on a bed.

### BACKGROUND

Devices and methods for delivering nebulized medicines are disclosed in International Application PCT/US13/39324, filed May 2, 2013, the complete disclosure of which is incorporated herein by reference. The devices disclosed therein include a positionable elongated member and a delivery tube, e.g., a length of flexible plastic tubing, disposed within the elongated member. Nebulized medicine is delivered from a medicine cup through the delivery tube to a patient.

### SUMMARY

Generally, this invention relates to devices and methods for delivery of a nebulized medicine to a patient. The devices disclosed herein may be configured for use in environments such as patients' homes, hospitals, doctors' offices, and nursing homes. In some embodiments, the devices are configured so that the patient may remain in his or her current location, positioning, and state (e.g., sitting in a chair or lying down, either asleep or awake) and the device can be transported and positioned in such a manner as not to disrupt or seriously change the patient's current state, minimizing the likelihood of sending the patient into an uncooperative state where treatments are less effective. In some cases, the devices can be used hands-free by suspending a portion of the device in a position to effectively deliver medication in close proximity to the nose and mouth of the patient without touching the patient. In preferred implementations, unlike mask delivery or intubation, delivery using the devices disclosed herein is substantially “contact-free,” without a mask or other portion of the device contacting the patient's nose or mouth.

In some implementations, the devices are used for delivery of nebulized medicines to infants in a hands-free manner, allowing nurses to administer treatments to multiple infants at one time.

In one aspect, the invention features an apparatus comprising an elongated member configured to support a delivery tube and maintain a distal end of the tube in a desired position; and a base configured to support a proximal end of the elongated member, the base including a chamber configured to receive a medicine cup containing a supply of medicine and position a first end of the medicine cup in fluid communication with a source of compressed gas and a second end of the medicine cup in fluid communication with the delivery tube.

Some implementations may include one or more of the following features. The elongated member may include an articulated arm assembly. Alternatively, the elongated member may include a flexible arm, e.g., a continuously flexible member such as “gooseneck” tubing. The proximal end of the elongated member may be configured to be received in the chamber. The proximal end of the elongated member may include a cylindrical portion configured to slide over and around the medicine cup. The proximal end of the elongated member may be configured to swivel within the chamber.

The apparatus may further include an elastomeric seal mounted on the base and having an opening configured to receive the first end of the medicine cup in sealing engagement. The elastomeric seal may include self-aligning surfaces configured to assist the user in positioning the first end of the medicine cup in the opening. The self-aligning surfaces may include a frustroconical outer surface, and a funnel-shaped inner surface.

The funnel-shaped inner surface may terminate at a bore extending through the elastomeric seal and providing fluid communication between the first end of the medicine cup and the source of compressed gas.

The proximal end of the elongated member may include a cylindrical sleeve configured to sealingly engage the second end of the medicine cup. The cylindrical sleeve may include self-aligning surfaces configured to assist the user in positioning the proximal end of the elongated member with respect to the medicine cup.

The base may define a fluid flow path for delivery of a compressed gas from the source of compressed gas to the medicine cup, which may be disposed entirely within the base. The base may include an inlet port configured for connection to the source of compressed gas.

In other aspects, the invention features methods of using the devices disclosed herein. In one aspect, the invention features a method comprising (a) placing a first end of a medicine cup in a chamber in the base of a device for delivery of nebulized medicine; (b) placing a proximal end of an elongated member of the device in the chamber such that the proximal end is supported by the base and a port of the proximal portion is in fluid communication with a second end of the medicine cup; and (c) connecting the base to a source of compressed gas. The base is configured such that the first end of the medicine cup is in fluid communication with the source of compressed gas and the elongated member is connected to a delivery tube such that the a second end of the medicine cup in fluid communication with the delivery tube.

The term “medicine cup,” as used herein, refers to a jet nebulizer cup which is designed such that when compressed gas is supplied to one end the cup atomizes, aerosolizes, vaporizes, or nebulizes the medication into a surrounding gas.

The terms nebulize, atomize, vaporize and aerosolize are used interchangeably to describe the process that occurs in the medicine cup.

The description may use perspective-based descriptions such as up/down, back/front, and top/bottom. Such descriptions are merely used to facilitate the discussion and are not intended to restrict the application of the embodiments of the present invention.

### DESCRIPTION OF THE DRAWINGS

FIG. 1 is a perspective view of a device according to one embodiment, in which the device includes an articulated arm.

FIG. 2 is an exploded perspective view of a portion of the device shown in FIG. 1.

FIG. 3 is a perspective view of the base and base joint assembly portions of the device, seen from the back.

FIG. 3A is a perspective view of the compressor connection components exploded from the base.

FIG. 4 is a bottom plan view of the base.

FIG. 4A is a cross-sectional view taken along line A-A in FIG. 4, showing the flow path of compressed air through the base.

FIG. 5 is a top plan view of the base and base joint assembly portions of the device.

FIG. 5A is a cross-sectional view taken along line A-A in FIG. 5, illustrating the nesting of the parts that are shown exploded in FIG. 2.

FIG. 6 is a partially exploded view, similar to the view in FIG. 3A but with the components partially assembled.

In the following detailed description, reference is made to the accompanying drawings in which are shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

### DETAILED DESCRIPTION

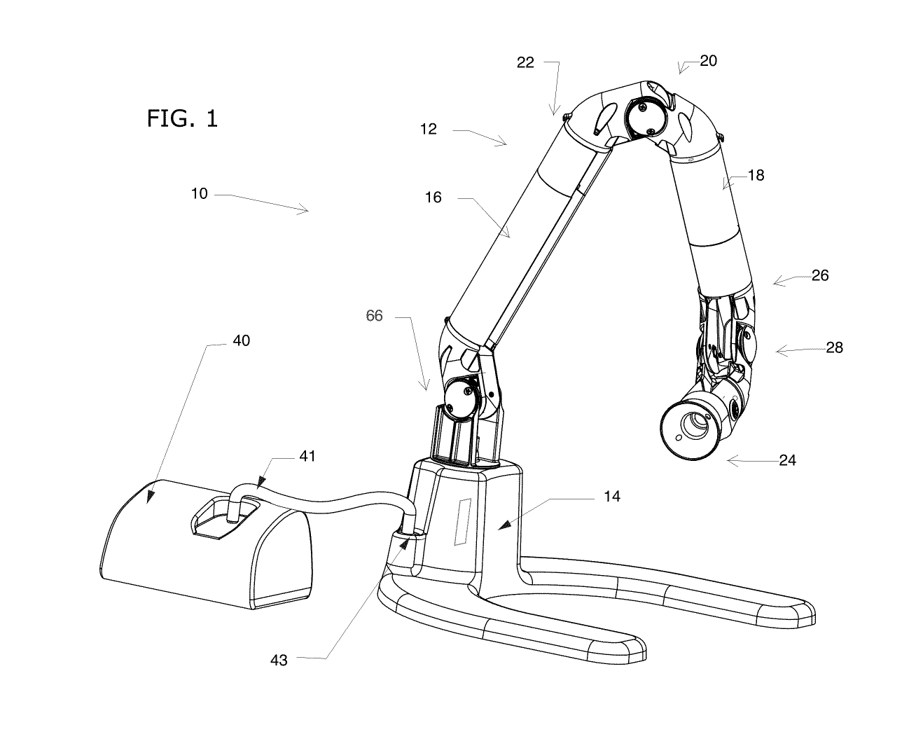

Referring to FIG. 1, in one embodiment a nebulized medicine delivery device 10 includes a positionable elongated member 12 supported by a base 14. In the embodiment shown in FIG. 1, the positionable elongated member 12 includes a first arm 16 which is coupled to the base 14 at its proximal end, and positionably coupled to a second arm 18 by an articulating joint 20 at its distal end 22. The articulating joint 20 allows the first arm 16 and second arm 18 to pivot about the axis of the hinge. Suitable structures for the articulating joint 20 are described in U.S. Ser. No. 13/934,408, Attorney Docket No. 000011-002US1, filed Jul. 3, 2013, the full disclosure of which is incorporated herein by reference.

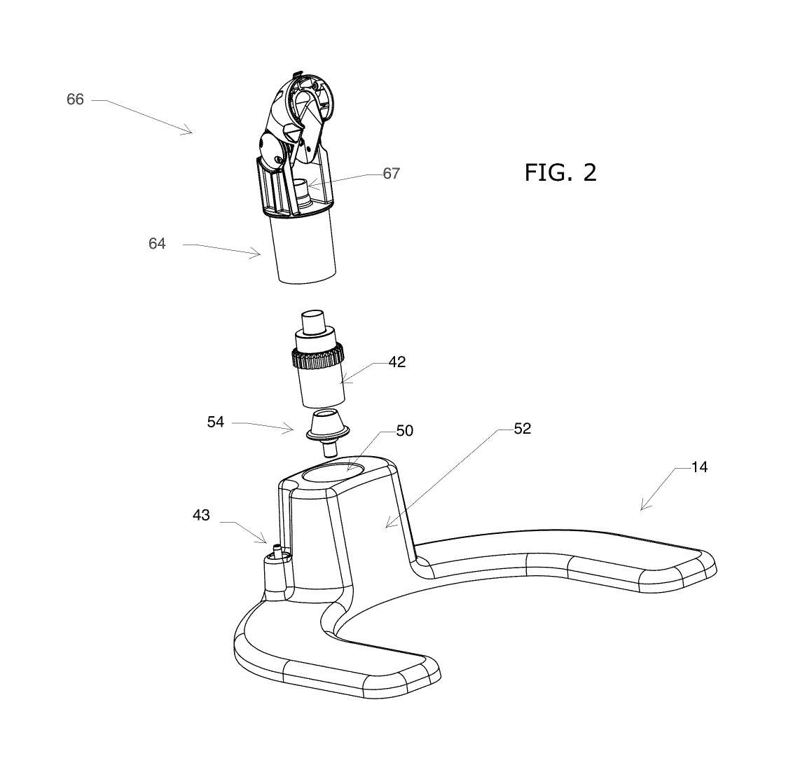

The device 10 also includes a compressor 40 which delivers compressed gas (e.g., air) via an air hose 41, to a medicine cup 42 (FIG. 2) which, when the device is in use, contains a supply of medicine. The air hose 41 is connected to the base 14 at a port 43. The medicine cup 42 is mounted within base 14, as will be described below in further detail, so as to hold the medicine cup in a stable, substantially vertical position during delivery of the medicine to enhance the effectiveness of the vaporizer. The compressor is connected to a power supply (not shown) or may be self-contained and battery powered. Medicine is nebulized in the medicine cup and then delivered to the patient via a delivery tube (note shown). The delivery tube is disposed in a channel that runs continuously through the first arm 16, the joint 20 and the second arm 18. This channel allows the tube to be held securely but removably within the positionable elongated member 12. Because the delivery tube is removable from the channel it can be easily replaced.

A nozzle 24 is mounted at the distal end 26 of the second arm, for delivery of the vaporized medicine to an area adjacent the patient's face. For example, delivery can be directed toward the patient's nose and mouth, so that the patient will receive the medicine regardless of whether she is breathing through her mouth or nose. Mounting of the nozzle may be by a pivotable connection 28, as shown, to provide fluid communication between the nozzle 24 and the delivery tube while allowing the position of the nozzle to be finely adjusted.

The proximal end of the first arm 16 is mounted on a base joint assembly 66, as will be discussed further below. Referring to FIG. 2, the base joint assembly 66 includes a tubing attachment port 43 on which the delivery tube can be easily mounted by the user.

Referring to FIGS. 2-6, the manner in which the medicine cup is mounted in the base 14 allows for easy removal and replacement of the medicine cup, easy connection of the medicine cup to the supply of compressed gas and the delivery tube, and stable positioning of the medicine cup during delivery.

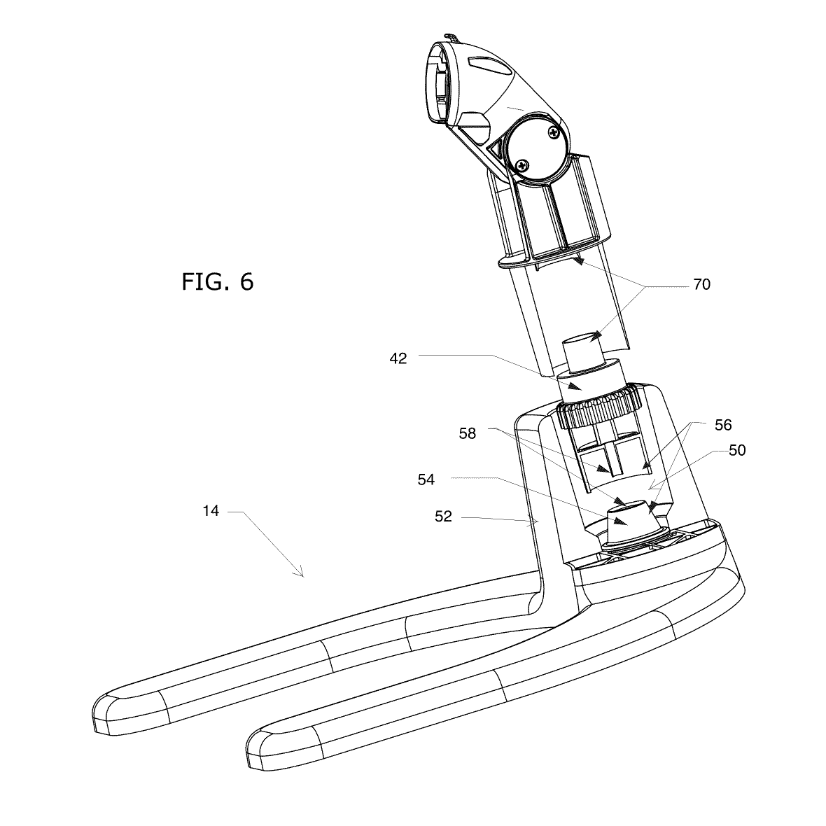

Referring to FIGS. 2 and 6, the medicine cup 42 is positioned in a cavity 50 defined in the upstanding portion 52 of the base 14. A self-centering elastomeric seal 54 is positioned between the medicine cup and the bottom of the cavity 50, and serves as a seat for the medicine cup. As shown in FIG. 6, the medicine cup and seal include self-aligning outer surfaces 56 (an inner female surface of the skirt of the medicine cup and an outer frustroconical male surface of the seal) and self-aligning inner surfaces 58 (a male protrusion of the medicine cup and a female funnel-shaped central opening in the seal.) The engagement of these cooperating surfaces allows the user to easily position the medicine cup in correct alignment with the flow path of the compressed air.

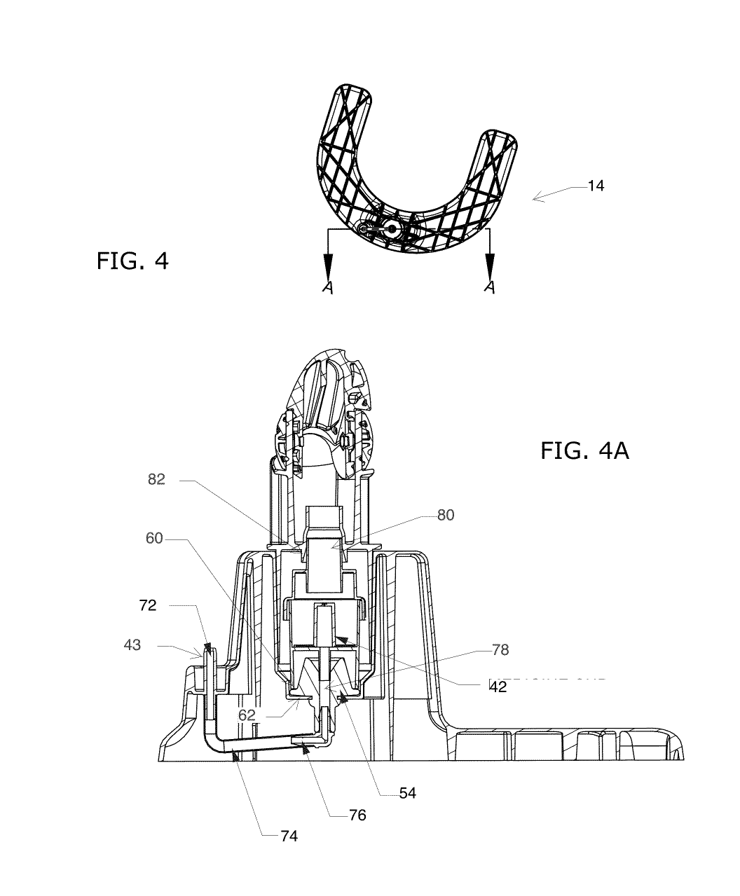

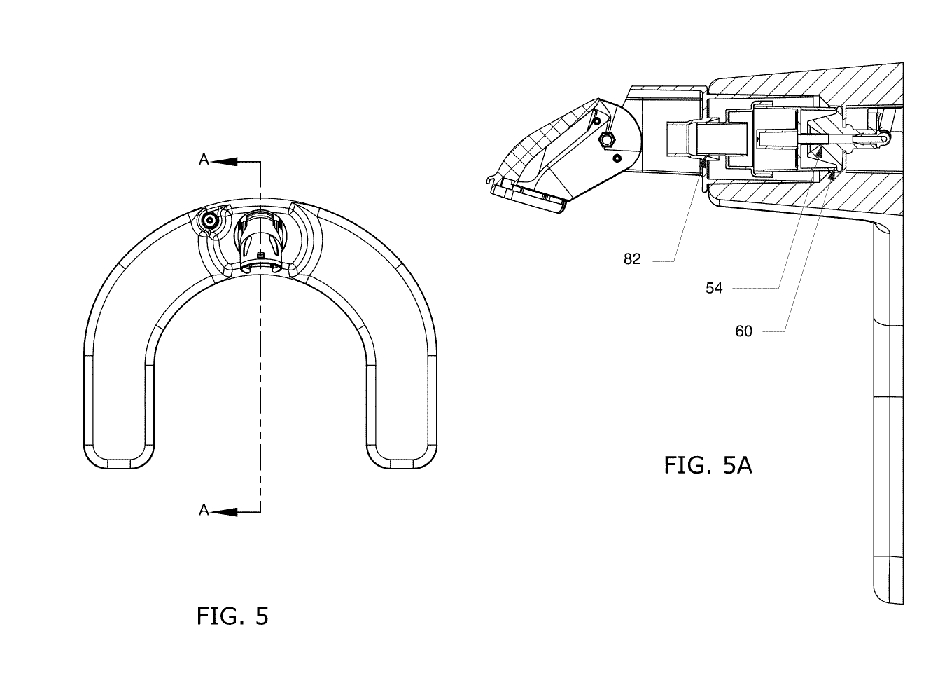

During assembly of the device, the seal is press-fit into an opening in the base, as can be seen in FIGS. 4A and 5A. The seal includes a rim 60 and lower surface 62 that seal against adjacent surfaces of the base, as shown in FIG. 4A. If necessary, the seal can be removed for cleaning or replacement; however, it is sufficiently durable that it can be left in place over multiple uses of the device.

After placing the medicine cup in the cavity 50, where it seats in seal 54, the user slides the lower cylindrical portion 64 (FIGS. 2, 6) of the base joint assembly 66 into the cavity 50 and over the medicine cup. Referring to FIG. 6, self-aligning surfaces 70 of the upper cylindrical portion 80 (FIG. 4A) of the medicine cup and skirt 82 (FIG. 4A) of the base joint assembly assist the user in positioning the base joint assembly over the medicine cup.

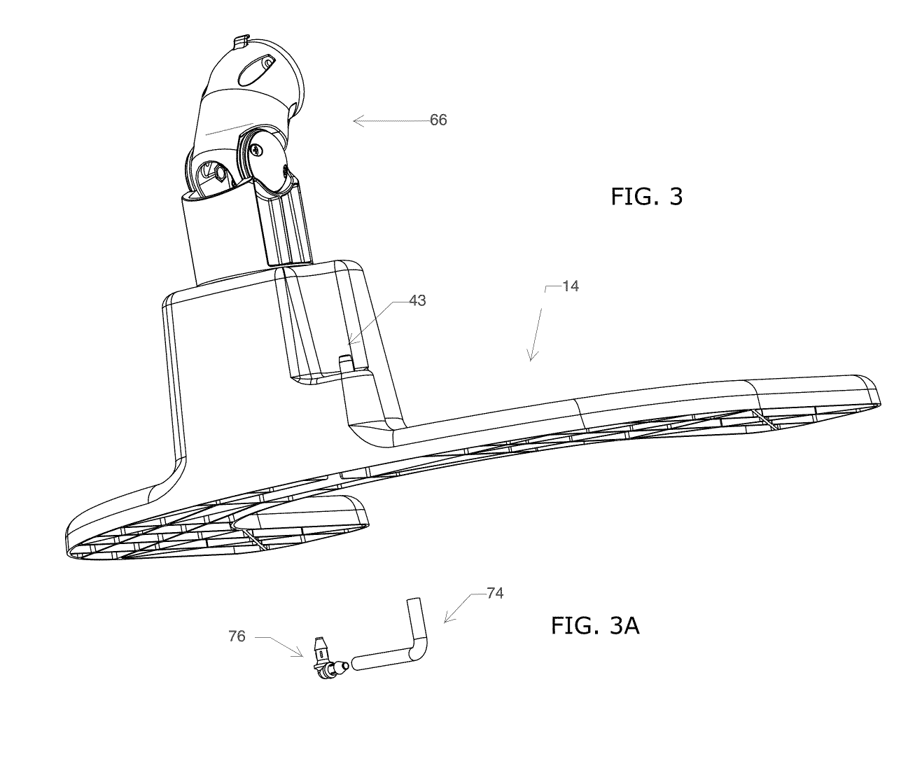

When base joint assembly has been positioned over the medicine cup, the device is ready for use, as shown in FIGS. 3, 4A and 5A, and a fluid flow path is established between the compressor and the delivery tube. This flow path is best seen in FIG. 4A, and consists of compressor connection port 43, inlet conduit 72, airflow connector 74 (also shown in FIG. 3A), and seal connection fitting 76 (FIG. 3A), which serves as an elbow joint and is in sealing engagement with the elastomeric seal 54 when the device is assembled. The flow path also includes bore 78 (FIG. 4A) in elastomeric seal 54 and the flow channel through the medicine cup, which terminates in upper cylindrical portion 80. Upper cylindrical portion 80 is in sealing engagement with skirt 82 of the base joint assembly, which is in fluid communication with the tubing attachment port 67 and thus the delivery tube, completing the flow path.

This manner of mounting the medicine cup in the device, and attaching the compressor to the device, allows the device to be easily assembled for use even under trying conditions, e.g., with a sick child or infant to attend to.

Preferably, the delivery device is configured to provide laminar flow of the vaporized medicine for a sufficient distance from the nozzle to allow the nozzle to be positioned a comfortable distance from the patient's face, i.e., to prevent the vaporized medicine from dispersing before it reaches the patient. It is also preferred that the device be configured so that beyond that distance (where the patient's face will be positioned) the laminar flow broadens out to a more dispersed stream or cloud. For example, the nozzle may provide about 4 to 8 inches of laminar flow, followed by about 2 to 4 inches of a broader, dispersed stream. In some implementations the laminar flow is visible, allowing easy visualization by a caregiver, clinician, or patient during delivery to the patient.

Several features of the device are believed to contribute to the laminar characteristics of the flow from the nozzle.

First, the delivery path of the vaporized medicine from the medicine cup to the nozzle is at least initially, in the vicinity of the medicine cup, straight or relatively straight. The lack of any abrupt changes of direction of flow is believed to contribute to the establishment of laminar flow.

Second, in preferred implementations the nozzle geometry is configured to enhance laminar flow, while limiting the distance of laminar flow to the expected distance between the nozzle and patient.

In use, the base 14 may be positioned on the floor or in any desired location, and the positionable elongated member 12 used to position the nozzle in various locations in space so that the nozzle is generally adjacent to the patient's face. The orientation of the nozzle can then be finely adjusted to the patient's face through the motion of the rotating ball joint, if a ball joint is provided. Adjustment of the positioning of the device can be facilitated by visualizing the laminar flow of the vaporized stream from the device, and further enhanced by turning on lighting, e.g., as disclosed in International Application No. PCT/US13/39324, filed May 2, 2013, the full disclosure of which is incorporated herein by reference. Once the position of the device has been adjusted in this manner, the device can be used hands-free, with the user needing only to monitor the patient and readjust the position of the nozzle if the patient moves out of position relative to the nozzle.

### OTHER EMBODIMENTS

A number of embodiments have been described. Nevertheless, it will be understood that various modifications may be made without departing from the spirit and scope of the disclosure.

For example, while asthma treatments have been discussed above, the devices and methods disclosed herein may be used for the delivery of any type of vaporized medicine, and in any type of treatment involving such delivery. Other treatments include treatment of cystic fibrosis, croup, pneumonia, and other respiratory conditions. The devices and methods may also be used to deliver gases to patients, for example for substantially contact-free, hands-free oxygen delivery to a patient that cannot tolerate a mask or nasal cannula due to facial trauma or other issues.

Moreover, while various applications have been discussed above, the devices and methods may be used in many other applications where non-contact and/or hands-free delivery would be advantageous. For example, the devices may be used by paramedics or other EMS personnel in situations where the caregiver should remain seated, e.g., in a moving ambulance or other transport.

While the device shown in FIG. 1 has only two arms, a third arm, or multiple articulated arms, could be interposed between the second arm and the nozzle if desired, to provide further articulation. In this case, the axes of rotation of the various joints between the arms may be oriented differently, or some or all of the joints may be capable of multi-axis movement.

If desired, the nozzle 24 may be omitted and the vaporized medicine simply delivered from the end of the delivery tube, or a different type of nozzle may be used, e.g., a spray head (not shown) which may be adjustable, e.g., in the manner of an adjustable garden hose sprayer. Suitable nozzles are disclosed, for example, in International Application No. PCT/US13/39324, filed May 2, 2013, the full disclosure of which was incorporated herein by reference above.

Moreover, in various embodiments, not shown or described, various aspects may be modified to accommodate extended positioning of the device to reach more and additional degrees of freedom to adapt to different environments. The shape, size and configuration shown in the drawings and discussed above are meant only as an example and are not intended to be limiting.

Accordingly, other embodiments are within the scope of the following claims.

What is claimed is:

1. An apparatus comprising:
an elongated member configured to support a delivery tube and maintain a distal end of the tube in a desired position; and
a base configured to support a proximal end of the elongated member, the base including a chamber configured to receive a medicine cup containing a supply of medicine and position a first end of the medicine cup in fluid communication with a source of compressed gas and a second end of the medicine cup in fluid communication with the delivery tube.

2. The apparatus of claim 1 wherein the elongated member comprises an articulated arm assembly.

3. The apparatus of claim 1 wherein the proximal end of the elongated member is configured to be received in the chamber.

4. The apparatus of claim 3 wherein the proximal end of the elongated member includes a cylindrical portion configured to slide over and around the medicine cup.

5. The apparatus of claim 1 further comprising an elastomeric seal mounted on the base and having an opening configured to receive the first end of the medicine cup in sealing engagement.

6. The apparatus of claim 5 wherein the elastomeric seal includes self-aligning surfaces configured to assist the user in positioning the first end of the medicine cup in the opening.

7. The apparatus of claim 1 wherein the proximal end of the elongated member comprises a cylindrical sleeve configured to sealingly engage the second end of the medicine cup.

8. The apparatus of claim 7 wherein the cylindrical sleeve includes self-aligning surfaces configured to assist the user in positioning the proximal end of the elongated member with respect to the medicine cup.

9. The apparatus of claim 1 wherein the base defines a fluid flow path for delivery of a compressed gas from the source of compressed gas to the medicine cup.

10. The apparatus of claim 9 wherein the flow path is disposed entirely within the base.

11. The apparatus of claim 3 wherein the proximal end of the elongated member is configured to swivel within the chamber.

12. The apparatus of claim 6 wherein the self-aligning surfaces include a frustroconical outer surface, and a funnel-shaped inner surface.

13. The apparatus of claim 12 wherein the elastomeric seal funnel-shaped inner surface terminates at a bore extending through the elastomeric seal and providing fluid communication between the first end of the medicine cup and the source of compressed gas.

14. The apparatus of claim 1 wherein the base includes an inlet port configured for connection to the source of compressed gas.

15. The apparatus of claim 1 wherein the elongated member comprises a flexible support arm that provides positional adjustment.

16. A method comprising:
placing a first end of a medicine cup in a chamber in the base of a device for delivery of nebulized medicine;
placing a proximal end of an elongated member of the device in the chamber such that the proximal end is supported by the base and a port of the proximal portion is in fluid communication with a second end of the medicine cup; and
connecting the base to a source of compressed gas;
wherein the base is configured such that the first end of the medicine cup is in fluid communication with the source of compressed gas and the elongated member is connected to a delivery tube such that the a second end of the medicine cup in fluid communication with the delivery tube.
